(19) United States
(12) Patent Application Publication
(10) Pub. No.: US 2011/0144475 A1
(43) Pub. Date: Jun. 16, 2011

(54) METHOD FOR A REGULATION OF A VASCULAR INTERVENTION, AND DEVICE TO REGULATE A VASCULAR INTERVENTION

(76) Inventors: Okan Ekinci, Uttenreuth (DE); Friedrich Fuchs, Roettenbach (DE); Markus Schmidt, Nuernberg (DE); Sebastian Schmidt, Weisendorf (DE); Anke Weissenborn, Weil am Rhein (DE)

(21) Appl. No.: 12/964,880

(22) Filed: Dec. 10, 2010

(30) Foreign Application Priority Data
Dec. 11, 2009 (DE) 10 2009 057 930.3

(51) Int. Cl. A61B 5/055

(52) U.S. Cl. 600/411

(57) ABSTRACT

In a method and apparatus to regulate a vessel intervention conducted with a vessel intervention unit, wherein an intervention region of the vessel intervention is at least partially imaged by means of an imaging monitoring unit, and a monitoring region to monitor the vessel intervention is selected, and the vessel intervention is at least partially automatically regulated in the intervention region dependent on at least one characteristic variable identified in the monitoring region.

### BACKGROUND OF THE INVENTION

1. Field of the Invention

The present invention concerns a method for regulating a vascular intervention, as well as a device to regulate a vascular intervention.

2. Description of the Prior Art

A method to manually adjust a vascular intervention is known in which the vascular intervention is conducted by means of a vascular intervention unit. The success of the vascular intervention is manually determined at a location of the intervention by a treating personnel (for example a physician) purely according to morphological criteria. For example, if a constriction in a vessel is expanded, an expansion of the vessel ensues up to an internal diameter of the vessel at this location of the intervention, for instance corresponding to an inner diameter in a further or preceding curve of the vessel. However, this can easily lead to an injury to the vessel since a risk of injury rises with the internal diameter of the vessel. An intervention region of the vascular intervention can hereby be depicted for the treating personnel by means of an ultrasound unit, a magnetic resonance unit, a computer tomography unit etc.

### SUMMARY OF THE INVENTION

An object of the present invention is to provide a method for a regulation of a vascular intervention that minimizes the danger of an injury to vessels during the vascular intervention.

The invention proceeds from a method for a regulation of a vascular intervention with a vascular intervention unit, wherein an intervention region of the vascular intervention is at least partially depicted by means of an imaging monitoring unit and a monitoring region is selected for monitoring of the vascular intervention.

In accordance with the invention, the vascular intervention in the intervention region is at least partially automatically regulated by means of at least one characteristic variable of a monitoring region. As used herein a vascular intervention in this context is a vascular procedure, in particular in a blood vessel, which can advantageously ensue by means of a catheter. A treatment of the blood vessel, in particular a vessel dilatation or a vessel embolization, is advantageously conducted by means of the vascular intervention. Furthermore, an intervention region means a region of the vessel in which an intervention treatment—for example a vessel embolization—ensues by means of which the blood flow in the vessel is at least partially suppressed or a vessel dilatation to a vessel expansion. A depiction of the intervention region is an imaging and/or an optical reproduction of an image of the region of the patient within which the intervention treatment takes place. A variation of a flow property (for example a perfusion) within the vessel and/or additional characteristic variables appearing to the man skilled in the art to be reasonable is advantageously identified by means of the characteristic variable. The characteristic variable can hereby be directly detected by a detection unit or can be determined from acquired data. The characteristic variable is additionally advantageously detected and/or determined within the monitoring region, wherein the monitoring region exhibits a sensitivity to the vascular intervention in the intervention region. Through the embodiment according to the invention, a duration and/or a strength of the vascular intervention can advantageously be adjusted depending on characteristic variable of the monitoring region, and therefore a danger of injury to the vessel to be treated can advantageously be at least reduced or prevented. In particular, a constriction in a vessel can be expanded until the point at which an at least partial (and particularly advantageously complete) functionality of the vessel is achieved, wherein the functionality can be determined using the characteristic variable. The regulation of the vascular intervention ensues automatically at least in part and particularly advantageously ensues automatically via a control unit. The regulation can additionally take place manually in part by the operator of the vascular intervention unit in that said operator is requested to change at least one parameter of the vascular intervention.

In accordance with the invention, the monitoring region can be arranged at least partially separate from the intervention region, so the treatment success can advantageously be detected by means of (for example) a perfusion property in the monitoring region. In particular, the monitoring region can be arranged at an easily accessible region of the patient that enables a simple monitoring. The monitoring region can be arranged (located) completely separate from the intervention region from a spatial standpoint. For example, the intervention region can be in a femoral region of a patient and the monitoring region is arranged in a foot region of the patient.

At least one vessel within the monitoring region is particularly advantageously supplied by a vessel of the intervention region that is to be treated. A dependency between the characteristic variable of the monitoring region and an intervention treatment in the intervention region can hereby be advantageously achieved, and an effective regulation of the vascular intervention can therefore be achieved. As used herein, “supplied” means that the content of the vessel (in particular blood) of the intervention region arrives in the at least one vessel of the monitoring region along a flow direction of the content of the vessel in a normal function of said vessel.

In an embodiment, the vascular intervention is conducted step by step in at least two intervention steps, whereby the characteristic variable is particularly advantageously determined in the monitoring region after each intervention step and therefore a particularly sensitive regulation of the vascular intervention can be achieved. A step by step vascular intervention is in particular advantageous given a dilatation intervention since here a detection of the characteristic variable in the monitoring region can only take place inadequately in the monitoring region during an intervention treatment. In particular, in the dilatation intervention a danger of injury to the vessel (in particular a rupture of the vessel) by a further expansion of the vessel can be at least reduced and/or prevented in that a treatment success is determined after each intervention step. A number of intervention steps is advantageously adapted to a duration and strength of the vascular intervention.

A change of the characteristic variable (and therefore of a functionality of the vessel) in the intervention region can advantageously be detected if a contrast agent is injected after at least one intervention step. The characteristic variable of the monitoring region can hereby be particularly advantageously determined from a temporal standpoint after an injection of the contrast agent.

If the characteristic variable of the monitoring region is determined at least partially in parallel with the vascular intervention, a treatment success of the vascular intervention can be determined simultaneously at least in part with the vascular intervention, and the vascular intervention can be ended immediately after the appearance of the success of the treatment. For example, a temporary closure of the vessel can be effectively adapted to a desired treatment success and an additional inflation of a balloon for the vessel closure can be prevented.

In a further embodiment of the invention, an intervention speed is at least partially adapted to the characteristic variable after a detection of the characteristic variable of the monitoring region. The intervention speed in this context is a speed for supplying an intervention substance (for example, for supplying a fluid in a balloon to seal the vessel) into the intervention region. A particularly exact adjustment of the intervention treatment can be achieved via this embodiment, and a risk of injury can thereby be minimized.

Furthermore, the vascular intervention can be automatically ended at least in part by means of a control unit after the characteristic value reaches a predetermined limit value. An optimally exact adjustment of the vascular intervention can be achieved, and additionally an imprecision due to a manual control and/or regulation by an operator can advantageously be prevented. The limit value advantageously indicates a variable that is coupled to a functionality of the vessel and/or a functionality of an organ dependent on the vessel. For example, in a dilatation treatment it is sufficient to expand the vessel to a width that enables a nearly complete perfusion, for example a width of approximately 80% of a comparable width in a region of the vessel directly adjoining an intervention region. A control unit is a unit that includes a processor for a data evaluation and/or a control and/or regulation of individual components of the method. In addition, the control unit can include additional components, for example a memory element.

Moreover, if the characteristic variable does not reach a predetermined limit value an additional intervention step is started, so an effective and in particular protective vascular intervention can be achieved in a step by step approach to the limit value.

Furthermore, the invention encompasses a device to regulate a vascular intervention, with a vascular intervention unit and an imaging monitoring unit to depict an intervention region of the vascular intervention.

The device in accordance with the invention as a control unit that conducts an at least partially automatic regulation of at least one intervention step of the vascular intervention. The vascular intervention unit also includes a catheter unit. An imaging monitoring unit is a unit that detects the vascular intervention and transfers it to an imaging medium (for example a monitor) so that a current treatment can be monitored by an operator of the device (for example a physician). The imaging monitoring unit is advantageously formed by a magnetic resonance unit, a computed tomography unit, an ultrasound unit and/or additional units appearing to be reasonable to the man skilled in the art. In this embodiment a fast adjustment and/or adaptation of the vascular intervention that is at least partially independent of decisions of an operator can advantageously be achieved by means of the control unit.

Furthermore, the device can include a detection unit that is provided to detect at least one characteristic variable of a monitoring region that is arranged at least partially separate from the intervention region. The progress and/or success of the vascular intervention can advantageously be detected depending on the characteristic variable of the monitoring region, and therefore a further treatment in the intervention region with a high risk of injury can advantageously be prevented. As used herein “provided” means specially equipped and/or specially designed. The detection unit is advantageously formed by a computed tomography unit, an ultrasound unit and/or additional units appearing to be reasonable to those skilled in the art.

The control unit particularly advantageously conducts the at least partially automatic regulation using the characteristic variable of the monitoring region, whereby a duration and/or strength of the vascular intervention can be adjusted depending on the characteristic variable of the monitoring region, and therefore a danger of an injury to the vessel to be treated can at least be reduced or prevented. In particular, a constriction in a vessel can be expanded until an at least partial (and particularly advantageously complete) functionality of the vessel is achieved, wherein the functionality is determined using the characteristic value. The regulation can thereby contain a request for a manual modification of a parameter of the vascular intervention and/or can particularly advantageously ensue completely independently by means of the control unit.

If the monitoring region has at least one vessel that is supplied by a vessel of the intervention region that is to be treated, a direct dependency between the characteristic variable of the monitoring region and an intervention treatment in the intervention region can hereby be advantageously achieved. An effective regulation of the vascular intervention can therefore be additionally achieved.

The device also can include a contrast agent unit with which a contrast agent can be injected, so a change to the characteristic variable (and therefore a functionality of the vessel) in the intervention region can advantageously be detected. Control of the contrast agent unit by the aforementioned control unit advantageously ensues.

Furthermore, the device can include a valve unit with which a feed of an intervention substance for the vessel unit can be controlled. A feed of an intervention substance for a vascular intervention is a feed of a gas and/or a liquid, for example to inflate a balloon for a vessel dilatation and/or a feed of particles for an at least partial vessel closure. An optimally exact substance feed can be achieved by means of the valve unit and a risk of injury during the treatment can be minimized.

If the control of the valve unit takes place by means of the control unit, an adjustment of the valve unit (and therefore the substance feed) can be adapted to currently-detected characteristic variables and advantageously can be matched to a success and/or progress of the vascular intervention.

The detection unit and/or the imaging monitoring unit can be formed by a magnetic resonance unit. The detection unit is advantageously fashioned as one part with the imaging monitoring unit. A time-consuming displacement of the patient between individual detection steps and individual monitoring steps can be advantageously avoided in that the magnetic resonance unit covers a large detection region. Different measurements in different measurement planes can additionally be realized essentially at the same time.

If the vascular intervention unit and/or the contrast agent unit and/or the valve unit are fashioned to be at least partially magnetic resonance-compatible, an unwanted interference of a magnetic resonance measurement with a detection of the characteristic variable of the monitoring region and/or with a monitoring of the intervention region can advantageously be avoided. An unwanted radiation exposure can be prevented, such that the depiction of the intervention region can take place at high quality. Furthermore, an interfering and in particular unwanted influence of a high magnetic resonance field on an effectiveness of the treatment can be prevented so that a radioactive contamination of patients and/or of an operator can be avoided.

### BRIEF DESCRIPTION OF THE DRAWINGS

FIG. 1 shows a device according to the invention in a schematic representation.

FIG. 2 shows a partial region of the device for a vascular intervention for a dilatation treatment in a schematic representation.

FIG. 3 shows a partial region of the device for an embolization treatment in a schematic representation.

FIG. 4 is a workflow diagram of a method according to the invention, subdivided into individual intervention steps.

FIG. 5 is a workflow diagram of the method of the vascular intervention and a simultaneous detection of a characteristic variable in a monitoring region.

### DESCRIPTION OF THE PREFERRED EMBODIMENTS

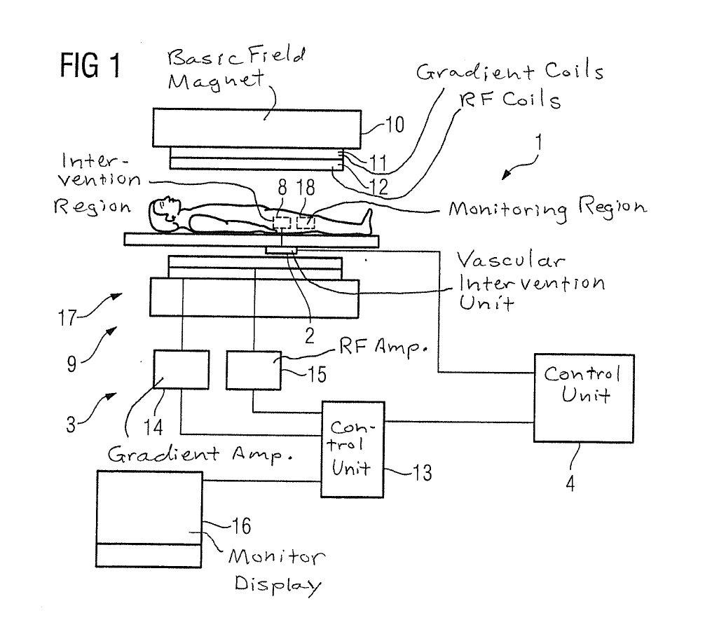

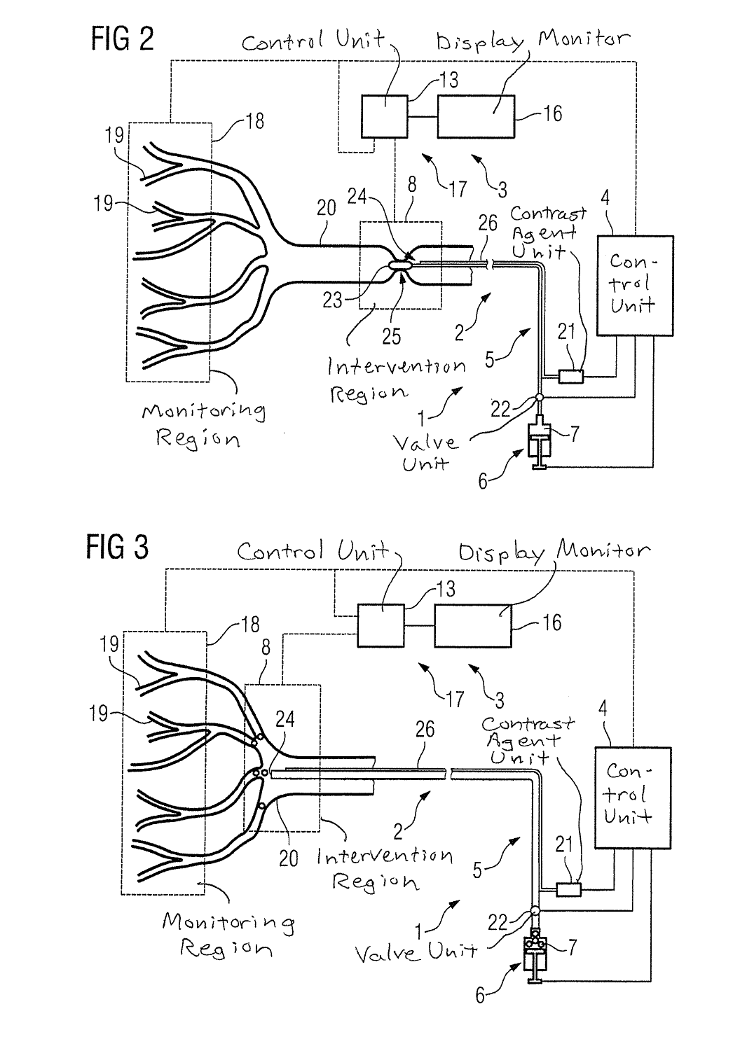

A device 1 to regulate a vascular intervention is shown in FIG. 1. The device 1 includes a vascular intervention unit 2, an imaging monitoring unit 3 and a control unit 4. The vascular intervention unit 2 has a catheter unit 5 and a substance unit 6 (FIGS. 2 and 3) by means of which an intervention substance 7 can be introduced into the catheter unit 5 (and therefore into an intervention region 8) or a substance can be conducted out of the intervention region 8 via the catheter unit 5 (FIGS. 2 and 3). In the exemplary embodiments regarding FIGS. 2 and 3 the substance unit 6 is formed by a syringe. The control unit 4 is designed for an at least partial automatic regulation of the vascular intervention in the operation of device 1.

The imaging monitoring unit 3 is fashioned to depict the intervention region 8 of the vascular intervention (FIG. 1). The intervention region 8 is or contains a region of a patient in which a vascular intervention treatment is conducted. For this purpose, the imaging monitoring unit 3 is formed by a magnetic resonance unit 9. The magnetic resonance unit 9 includes a basic field magnet 10 to generate a strong and constant magnetic field. Furthermore, the magnetic resonance unit 9 possesses gradient coils 11 that are provided to generate a linear gradient field and radio-frequency (RF) coils 12. The gradient coils 11 are controlled by a control unit 13 of the magnetic resonance unit 9 via a gradient amplifier 14. The radio-frequency coils 12 are controlled by the control unit 13 via a radio-frequency amplifier 15 and are additionally provided to receive a magnetic resonance signal. The magnetic resonance unit 9 also has a monitor display 16 by means of which the magnetic resonance exposures and/or results evaluated by the control unit 13 can be presented (FIG. 1).

Furthermore, the device 1 includes a detection unit 17 that is likewise formed by the magnetic resonance unit 9 and thus is fashioned in one piece with the imaging monitoring unit 3. In the operation of the device 1 the detection unit 17 is provided to detect at least one characteristic variable of a monitoring region 18 in a patient. For this purpose, the monitoring region 18 is located spatially separate from the intervention region 8. For an advantageous detection of the characteristic variable that delivers or provides at least one item of information about the intervention treatment in the intervention region 8, the monitoring region 18 contains at least one vessel 19 that is supplied by a vessel 20 of the intervention region 8 that is to be treated (FIGS. 2 and 3). For example, the intervention region 8 can be a partial region of a thigh and the vessel 20 to be treated can represent a blood vessel in the thigh. The monitoring region 18 associated with this can be, for example, a partial region of a foot with at least one blood vessel that is associated therewith. In alternative embodiments, the imaging monitoring unit 3 and/or the detection unit 17 can be formed by a computed tomography unit, an x-ray fluoroscopy unit, an angiography unit, an ultrasound unit, etc.

The device 1 furthermore has a contrast agent unit 21 with which a contrast agent can be injected and a valve unit 22 that controls the substance feed of the intervention substance 7 for the vascular intervention (FIGS. 1 through 3). For a disruption-free operation of the device 1, the vascular intervention unit 2, the contrast agent unit 21 and the valve unit 22 are fashioned to be magnetic resonance-compatible. The control unit 4 is connected in a data exchange with the individual units and elements of the device 1 via a data line.

Two different embodiments of the device to regulate the vascular intervention are shown in FIGS. 2 and 3, wherein the difference between the two embodiments is in a design of the catheter unit 5. In FIG. 2 the catheter unit 5 has an elastic element 23 (for example a balloon) impermeable to particle exchange for (for example) a dilatation treatment. The elastic element 23 impermeable to particle exchange is arranged at one end 24 of the catheter unit 5 that is inserted into the patient and is connected with the catheter unit 5 such that the elastic element 23 impermeable to a particle exchange can be filled or emptied with the intervention substance 7 by the substance unit 6 exclusively via the catheter unit 5. The catheter unit 5 from FIG. 3 is provided for an embolization treatment and is fashioned open at the end 24 inserted into the patient so that the intervention substance 7 (formed by embolization elements) can be introduced into the vessel 20.

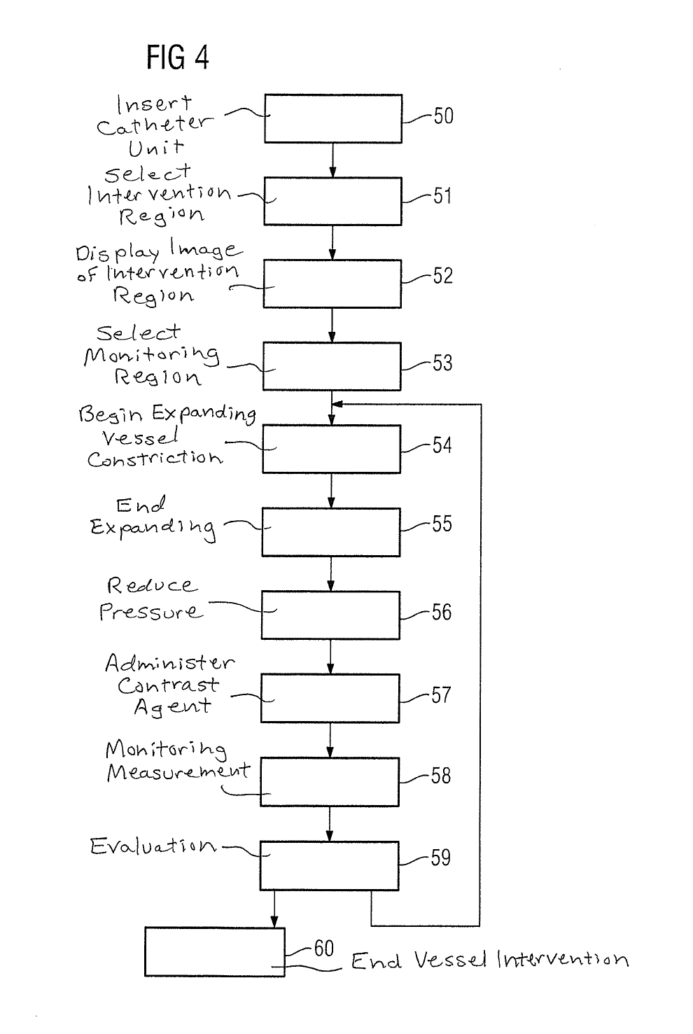

A method to regulate the vessel intervention by means of a vessel intervention unit 2 is depicted in FIG. 4. The method includes a step by step vessel intervention and here is explained in detail as an example using the exemplary embodiment of a dilatation treatment from FIG. 2. After an insertion 50 of the catheter unit 5 into the vessel 20 of the patient, a selection 51 of the intervention region 8 initially ensues. By means of the imaging monitoring unit 3 (in particular the magnetic resonance unit 9) the intervention region 8 is subsequently presented to a personnel (for example a physician) operating the device 1. The intervention region 8 comprises a region, in particular a slice within the patient, in which the intervention treatment is implemented and its image is presented by means of the magnetic resonance unit 9. For a presentation and/or imaging of the intervention region 8, magnetic resonance exposures of at least a partial region of the intervention region 8 are initially acquired and these are subsequently evaluated by the control unit 13 within the magnetic resonance unit 9 and are thereupon presented via the monitor display 16. Furthermore, it is also possible for the introduction 50 of the catheter unit 5 into the vessel 20 of the patient to already be shown or imaged by means of the magnetic resonance unit 9.

After the presentation 52 and/or imaging of the intervention region 8, a monitoring region 18 for a monitoring of the vessel intervention is selected that, in the present exemplary embodiments, is arranged completely separate from the intervention region 8 (FIGS. 2 and 4). A selection 53 of the monitoring region 18 is likewise conducted by the personnel conducting the intervention treatment. For this purpose, an enlarged region is initially acquired by means of the magnetic resonance unit 9 and depicted via the monitor display 16. The monitoring region 18 comprises a region, in particular a slice within the patient, whose image is likewise shown by means of the magnetic resonance unit 9.

After the monitoring region 18 is selected, the actual vessel intervention treatment begins that is subdivided into individual intervention steps, wherein all intervention steps are imaged by means of the magnetic resonance unit 9 via its monitor display 16. In the following the step by step vessel intervention is shown in detail together with a dilatation treatment of a vessel constriction 25 (FIG. 2). However, an application of the step by step vessel intervention in an embolization treatment (FIG. 3) is possible at any time. The first intervention step 54 of the step by step vessel intervention is started by activation of the substance unit 6 by the personnel operating the device 1. The intervention substance 7 (that can be formed by a gas, a gas mixture and/or a liquid) is introduced into the catheter unit 5 by means of the substance unit 6. A pressure is thereby generated in the substance unit 6 that leads via the catheter unit 5 to an inflation of the elastic element 23 that is impermeable to a particle exchange. The valve unit 22 with a valve with which a filling speed of the intervention substance 7 and/or a pressure in the catheter unit 5 (and therefore in the elastic element 23 impermeable to a particle exchange) is controlled is arranged between the substance unit 6 and the catheter unit 5. The valve unit 22 is controlled by the control unit 4.

In the intervention step 54, a pressure in the elastic element 23 impermeable to a particle exchange is built up via the catheter unit 5 to expand the vessel constriction. After the pressure built up in the catheter unit 5 has reached a predetermined value, and therefore after the vessel 20 has expanded at the constriction 25, an end 55 of the intervention step 54 takes place. The intervention step 54 is then endued automatically by the control unit 4. For this the valve of the valve unit 22, controlled by the control unit 4, is closed so that a further substance feed into the catheter unit 5 is prevented. In a subsequent method step pressure reduction (controlled by the control unit 4) will discharge the pressure from the elastic element 23 impermeable to a particle exchange and the catheter unit 5 via the valve unit 22, and the blood flow constriction 25 is released. For a pressure monitoring in the catheter unit 5 the control unit 4 includes a pressure sensor (not shown in detail).

As soon as the pressure in the catheter unit 5 has dissipated, a contrast agent administration 57 is started and a contrast agent is injected into the vessel 20 by means of the contrast agent unit 21. For this the contrast agent unit 21 includes an introduction means 26 that runs essentially in parallel with the catheter unit 5, next to this along the vessel 20 up to the treatment region of the vessel intervention so that the injected contrast agent is introduced directly into the treatment region. The contrast agent can be a contrast agent containing gadolinium, a contrast agent containing iron, a contrast agent containing fluorine or a hyperpolarized contrast agent for magnetic resonance measurements. In principle, a use of a contrast agent containing iodine is also conceivable, in particular if the acquisition unit comprises a computer tomography unit.

After the contrast agent injection, a monitoring measurement 58 ensues to detect the characteristic variable in the monitoring region 18 by means of the magnetic resonance unit 9. The data acquired by means of the monitoring measurement 58 are relayed to the control unit 4 and there are evaluated in an evaluation step 59. At the same time the data can be evaluated within the magnetic resonance unit 9 for an optical depiction and/or presentation of the monitoring region 18 in addition to the presentation of the intervention region 8. In the control unit 4 the characteristic variables that represent a value for a perfusion of the monitored vessel 19 are determined from the measured data. For example, a perfusion degree at the vessel constriction 25 can be concluded using a contrast agent concentration and/or a change of the contrast agent concentration. This value also reflects a functionality of the constriction 25 treated by means of the vessel intervention since an at least partial perfusion of the vessels 19 can only be present in the monitoring region 18 if an at least partial treatment success at the constriction 25 has already occurred. The degree of the perfusion in the monitoring region 18 and/or in the monitored vessel 19 additionally indicates how large a proportional factor of the recovered functionality is measured relative to the normal functionality of the treated vessel 20. The characteristic variable is additionally compared with a predetermined limit value within the evaluation step 59. The predetermined limit value indicates a minimum value of the recovered functionality that, for example, is sufficient for a full functionality of an organ dependent on the vessel 19. The predetermined limit value can be individually adapted to the respective vessel intervention and/or to the patient by the treating personnel before an intervention treatment.

If it is determined by the control unit in the evaluation step 59 that the determined characteristic variable is smaller than the predetermined limit value, an additional intervention step 54 is automatically started by the control unit 4. For this purpose, the valve 22 (controlled by the control unit 4) is opened again so that a pressure can build in the intervention region 8 (in particular the vessel constriction 25) via the catheter unit 5 and the elastic element 23 impermeable to a particle exchange. A workflow of the second intervention step 54 and possible additional intervention steps 54 proceeds analogous to the description of the first intervention step 54, with the exception that a greater pressure is respectively built up in the catheter unit 5 and the elastic element 23 impermeable to a particle exchange than was the case in the respective, preceding intervention step 54. A pressure build-up for the feed of the intervention substance 7 can furthermore be implemented manually by the personnel operating the device 1, or can ensue automatically, at least in part, via the control unit 4. The individual intervention steps 54 with the method steps 55, 56, 57, 58, 59 interposed between them repeat within a short time period so that, upon activation of the substance unit 6, the operator (in particular the physician) only experiences a different counter-pressure upon pressing a piston of the substance unit 6, wherein the different counter-force indicates the different method steps 55, 56, 57, 58, 59.

As soon as the determined characteristic variable has reached and/or exceeded the predetermined limit value in the evaluation step 59, an end 60 of the vessel intervention ensues and the control unit 4 automatically ends the vessel intervention. In this way an over-expansion of the vessel 20 to be treated is advantageously prevented and therefore a danger of injury to a vessel is minimized. The vessel intervention is additionally likewise ended automatically by the control unit 4 as soon as the pressure in the catheter unit 5 and in the elastic element 23 impermeable to a particle exchange assumes a maximum limit value that would lead to an over-expansion of the vessel 20 and therefore would significantly increase a risk of an injury to the vessel. Instead of an automatic ending 60 of the vessel intervention by the control unit 4, it is additionally possible for the control unit 4 to request (prompt) that the operator end the vessel intervention upon reaching the predetermined limit value for the characteristic variable and/or upon reaching the maximum limit value for the pressure in the catheter unit 5 and/or in the elastic element 23 impermeable to a particle exchange.

Alternatively, the detection of the characteristic variable can ensue by using a flow-sensitive magnetic resonance frequency and/or an arterial spin labeling (ASL) measurement so that the method step of the contrast agent administration 57 can be omitted. Furthermore, in an alternative embodiment of the invention it can be provided that the monitoring region 18 and/or the intervention region 8 is monitored by means of a computer tomography unit and/or an angiography unit and/or an x-ray fluoroscopy unit and/or an ultrasound unit instead of the magnetic resonance unit 9.

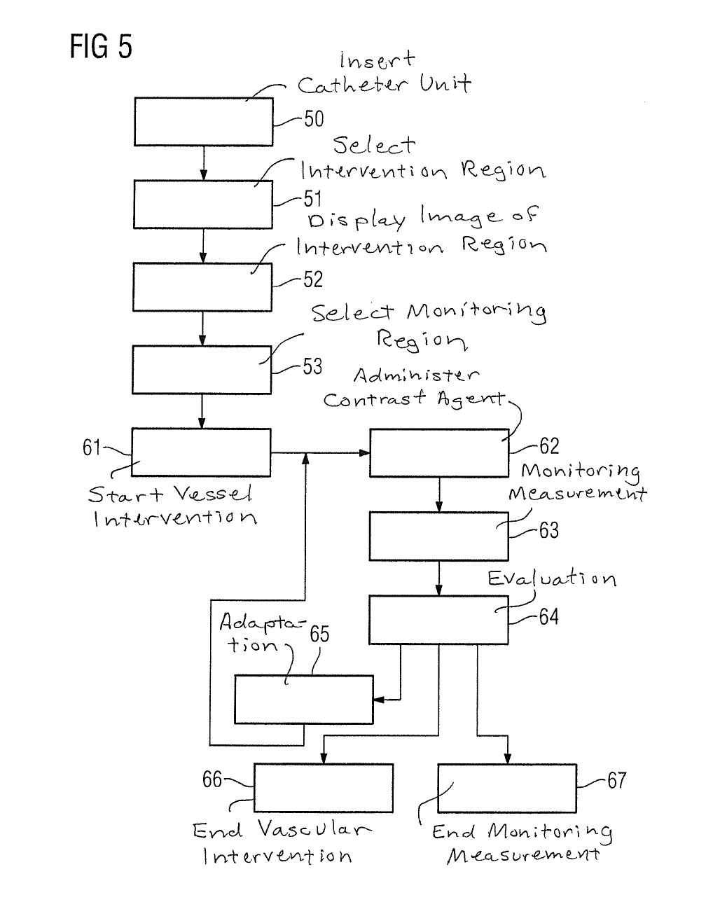

As an alternative to the step by step vascular intervention, in FIG. 5 a further embodiment of the method to regulate the vascular intervention is shown in which the characteristic variable is detected and/or determined in parallel with the vascular intervention. The method from FIG. 5 is explained in detail together with the exemplary embodiment of the embolization treatment from FIG. 3. However, an application of the method from FIG. 5 in a dilatation treatment (FIG. 2) is possible at any time. An introduction 50 of the catheter unit 5 and a selection 51 and/or presentation 52 of the intervention region 18 ensue in a manner analogous to the embodiments regarding FIG. 4. Furthermore, the selection 53 of the monitoring region 18 likewise ensues in a manner analogous to the embodiments regarding FIG. 4.

In FIG. 5 the vascular intervention is an embolization treatment in order to close at least one partial region of the vessel 20 in the intervention region 8, for example in order to quell a bleeding and/or to suppress a vessel supply to a tumor etc. After a start of an intervention step 61, an intervention substance 7 in the form of particles (for example small coils) is introduced by the substance unit 6 into the vessel 20 via the catheter unit 5. For this a pressure in the substance unit 6 and the catheter unit 5 is built up (analogous to the method in FIG. 4) in the substance unit 6 and the catheter unit 5 by the operator operating a piston of the substance unit 6. To introduce the particles, the catheter unit 5 is open at the inserted end 24. The particles are dimensioned such that they close the vessels at the desired vessel branch, wherein here a lumen and/or a cross section of the branched vessel is smaller than a lumen and/or a cross section of the vessel 20 before the branch. In order to prevent an unwanted vessel closure at further branches of the vessel 20, the particles are only brought into the vessel 20 by means of the catheter unit 5 just before the branch.

The monitoring of the monitoring region 18 also starts with the intervention step 61. A contrast agent administration 62 by means of the contrast agent unit 21 is initially started in a manner analogous to the method step 57 in the embodiments regarding FIG. 4. The contrast agent administration 62 can hereby ensue continuously or in individual administration steps. A monitoring measurement 63 is started after the beginning of the contrast agent administration 62. In order to avoid a long wait time for a propagation of the contrast agent, the contrast agent is also only introduced into the vessel 20 directly before the desired vessel closure. At the same time a monitoring measurement 63 is started that continuously acquires data in the monitoring region 18 to determine the characteristic variable in parallel with the vascular intervention. In an evaluation step 64, one or more characteristic variables are subsequently determined in the control unit 4 from the acquired data. A degree of perfusion at the point of the vessel closure is concluded using a contrast agent concentration and/or a change of the contrast agent concentration. In principle, it is additionally possible to implement a comparison measurement with a previous contrast agent administration before the beginning of the vascular intervention in order to detect a difference in the contrast agent concentration.

An evaluation of the measured data analogous to the statements regarding FIG. 4 additionally ensues in the control unit 4 in the evaluation step 64. Here as well a comparison of the characteristic variable with a previously determined limit value (for example a limit value for a maximum tolerable remaining perfusion in the partial region of the vessel 19 that is separated from a perfusion) ensues within the evaluation. As long as the detected characteristic variable lies above the predetermined limit value, particles are continuously introduced into the vessel 20 via the catheter unit 5 by means of the substance unit 6. Given a further supply of particles into the vessel 20, the characteristic variable continuously approaches the predetermined limit value until this is reached since a closure of partial regions of the vessel 20 increases. The possibility hereby exists that the control unit 4 is adapts an intervention speed to the determined characteristic variable in an adaptation step 65 and thus a speed of a substance feed is reduced. The adaptation of the intervention speed ensues via the valve unit 22, wherein the valve unit 22 is controlled by the control unit 4. At the same time an adaptation of the intervention speed by means of the substance unit 6 is also possible, wherein here as well a control of the substance unit 6 advantageously ensues by means of the control unit 4. In spite of the adaptation, the method steps 62, 63, 64, 65 are repeated continuously during the vascular intervention so that an optimally exact adaptation of the vascular intervention to a current treatment success is possible.

As soon as it is clear from the characteristic variables that the desired partial vessel region is closed and/or a perfusion of the partial region is below the predetermined limit value or has been entirely suppressed, the end 66 of the vascular intervention and an end 67 of the monitoring measurement 62 take place. The end 66 of the vascular intervention ensues automatically by means of the control unit 4. At the same time the detection of the characteristic variable by means of the control unit 4 is also ended.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method to regulate a vessel intervention in the body of a patient, comprising the steps of:
implementing a vessel intervention in the body of a patient using a vessel intervention unit that interacts with a vessel in the body of the patient in an intervention region in the body of a patient;
with an imaging monitoring unit, generating and displaying an image of at least a portion of said intervention region;
with said imaging monitoring unit, also generating an image of a monitoring region in the patient, said image of said monitoring region comprising monitoring image contents; and
at least partially automatically controlling operation of said intervention unit, to at least partially automatically regulate said vessel intervention, dependent on at least one characteristic variable of said monitoring region derived from said monitoring region contents.

2. A method as claimed in claim 1 comprising locating said monitoring region spatially separate from said intervention region.

3. A method as claimed in claim 1 comprising selecting said monitoring region as a region in the body of the patient that contains at least one vessel that is supplied by a vessel in the intervention region that is subjected to said vessel intervention.

4. A method as claimed in claim 1 comprising implementing said vessel intervention in at least two successive intervention steps.

5. A method as claimed in claim 4 comprising injecting contrast agent into the body of the patient after at least one of said intervention steps.

6. A method as claimed in claim 5 comprising deriving said at least one characteristic variable after administration of the contrast agent.

7. A method as claimed in claim 1 comprising determining said characteristic variable at least partially in parallel with said vessel intervention.

8. A method as claimed in claim 1 comprising adapting an intervention speed of said vessel intervention dependent on said characteristic variable.

9. A method as claimed in claim 1 comprising controlling said vessel intervention unit from a computerized control unit and providing said characteristic variable to said computerized control unit and, from said computerized control unit, automatically controlling said vessel intervention unit to end said vessel intervention when said characteristic variable reaches a predetermined limit value.

10. A method as claimed in claim 1 comprising implementing said vessel intervention in a plurality of intervention steps, monitoring said characteristic variable with respect to a predetermined limit value during said vessel intervention, and beginning at least one of said intervention steps as long as said characteristic value has not reached said predetermined limit value.

11. A device to regulate a vessel intervention in the body of a patient, comprising:
a vessel intervention unit configured to implement a vessel intervention in the body of a patient by interacting with a vessel in the body of the patient in an intervention region in the body of a patient;
an imaging monitoring unit that generates and displays an image of at least a portion of said intervention region;
said imaging monitoring unit also generating an image of a monitoring region in the patient, said image of said monitoring region comprising monitoring image contents; and
a control unit configured to at least partially automatically control operation of said intervention unit, to at least partially automatically regulate said vessel intervention, dependent on at least one characteristic variable of said monitoring region derived from said monitoring region contents.

12. A device as claimed in claim 11 wherein said imaging monitoring unit obtains said monitoring region at a location in the body of the patient that is spatially separate from said intervention region.

13. A device as claimed in claim 11 wherein said imaging monitoring unit obtains said monitoring region at a location in the body of the patient that contains at least one vessel that is supplied by a vessel in the intervention region that is subjected to said vessel intervention.

14. A device as claimed in claim 11 wherein said vessel intervention unit is configured to implement said vessel intervention in at least two successive intervention steps.

15. A device as claimed in claim 14 comprising a contrast agent unit that injects contrast agent into the body of the patient after at least one of said intervention steps.

16. A device as claimed in claim 15 wherein said control unit is configured to derive said at least one characteristic variable after administration of the contrast agent.

17. A device as claimed in claim 11 wherein said control unit is configured to determine said characteristic variable at least partially in parallel with said vessel intervention.

18. A device as claimed in claim 11 wherein said control unit is configured to adapt an intervention speed of said vessel intervention dependent on said characteristic variable.

19. A device as claimed in claim 11 wherein said computerized control unit is configured to automatically control said vessel intervention unit to end said vessel intervention when said characteristic variable reaches a predetermined limit value.

20. A device as claimed in claim 11 wherein said vessel intervention is configured to implement said vessel intervention in a plurality of intervention steps, and wherein said control unit is configured to monitor said characteristic variable with respect to a predetermined limit value during said vessel intervention, and to operate said vessel intervention unit to begin at least one of said intervention steps as long as said characteristic value has not reached said predetermined limit value.

21. A device as claimed in claim 11 wherein said imaging monitoring unit is a component of a magnetic resonance imaging apparatus.

22. A device as claimed in claim 21 wherein said vessel intervention unit is comprised of magnetic resonance-compatible material.
